(19) United States
(12) Patent Application Publication
(10) Pub. No.: US 2009/0240220 A1
(43) Pub. Date: Sep. 24, 2009

(54) Compressed Substrates Configured to Deliver Active Agents

(75) Inventors: John Gavin MacDonald, Decatur, GA (US); Andrew Mark Long, Appleton, WI (US); Mary L. McDaniel, Appleton, WI (US); Jin Heo, Gyeonggi-do (KR); Jaeho Kim, Roswell, GA (US); Lindsey Marie Jain, Winona Lake, IN (US)

Correspondence Address: DORITY & MANNING, P.A., POST OFFICE BOX 1449, GREENVILLE, SC, 29602-1449, US

(73) Assignee: Kimberly-Clark Worldwide, Inc, Neenah, WI (US)

(21) Appl. No.: 12/052,327

(22) Filed: Mar. 20, 2008

(51) Int. Cl. A61F 13/42; A61F 13/53

(52) U.S. Cl. 604/361; 604/380

(57) ABSTRACT

A compressed substrate having an altered upper surface is generally disclosed. The compressed substrate is configured to expand in the z-direction upon contact with a liquid to form an expanded substrate without substantially expanding in either the x-direction or the y-direction. The altered upper surface of the expanded substrate has an expanded surface area that is at least about 110% of the initial surface area of the compressed substrate. The compressed substrate is constructed from a compression molded web and includes an active agent. The compressed substrate can be included within the construction of an absorbent article to transfer the active agent to a wearer. The upper surface of a compressed substrate can be altered after formation of the compressed substrate. Alternatively, the upper surface of the compressed substrate can be altered during the compression process.

### BACKGROUND OF THE INVENTION

Many articles intended for personal wear (e.g., such as diapers, training pants, feminine hygiene products, adult incontinence products, bandages, medical garments and the like) are designed absorb moisture from liquid body exudates including urine, menses, blood, etc. and pull moisture away from the wearer to reduce skin irritation caused by prolonged wetness exposure. However, by making absorbent articles so absorbent, it is difficult for the wearer to realize that an insult of the article has occurred. In some instances, it may be desirable to give a signal (e.g., an uncomfortable and/or wet feeling against the skin) to alert the wearer that the act of urination has occurred. On the other hand, there is a counter-balancing concern about the possibility of skin irritations and rashes caused by prolonged wetness against the skin if the articles are less absorbent to allow the child to sense wetness.

To this end, some prior articles intended for personal wear during toilet training include means for alerting a child to urination without leaving a substantial amount of wetness against the skin. One example of training pants intended to provide a sensory indication of urination includes an element that changes size after urination (e.g., expanding upon wetting). However, such elements are typically surrounded by highly absorbent structures (sometimes referred to as absorbent cores) which compete for and may draw urine away from the element, thereby prolonging or otherwise inhibiting the expansion thereof and diminishing its potential training effectiveness. Also, superabsorbent material (SAM) which is used to make the highly absorbent structures of such articles expands upon absorbing urine. Such expansion may mask or otherwise cushion the feeling of the expanded sensory element, thus making it difficult for the wearer to sense the intended signal. Additionally, the expanding element can expand not only in the direction toward the crotch of the wearer, but also can expand in the plane of the article. This expansion in the plane of the absorbent article can result in increased pressure on the saturated absorbent core, making the absorption and retention of the absorbed liquid more difficult. Thus, the absorbing capacity of the absorbent core can be diminished, which may result in unwanted wetness remaining on the skin of the wearer and/or liquid leaking out of the absorbent article.

Consequently, while there has been progress in the design of personal absorbent articles capable of alerting a wearer to a release of liquid body exudates, there continues to be a need for improvements in such articles.

### SUMMARY OF THE INVENTION

Objects and advantages of the invention will be set forth in part in the following description, or may be obvious from the description, or may be learned through practice of the invention.

The present invention is generally directed to a compressed substrate having an altered upper surface. The compressed substrate is configured to expand in the z-direction upon contact with a liquid to form an expanded substrate without substantially expanding in either the x-direction or the y-direction. The altered upper surface of the expanded substrate has an expanded surface area that is at least about 110% of the initial surface area of the compressed substrate. The compressed substrate is constructed from a compression molded web and includes an active agent. The compressed substrate can be included within the construction of an absorbent article to transfer the active agent to a wearer. The upper surface of a compressed substrate can be altered after formation of the compressed substrate. Alternatively, the upper surface of the compressed substrate can be altered during the compression process.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1A shows an exemplary compressed substrate in its compressed state;

FIG. 1B shows the exemplary compressed substrate of FIG. 1A in its expanded state;

FIGS. 2A and 2B show an exemplary compressed substrate having a concave upper surface in both its compressed and expanded states, respectfully;

FIGS. 3A and 3B show an exemplary compressed substrate having a plurality of apertures in its upper surface in both its compressed and expanded states, respectfully;

FIGS. 4A and 4B show an exemplary compressed substrate having a convex upper surface in both its compressed and expanded states, respectfully;

FIGS. 5A and 5B show an exemplary compressed substrate having a plurality of protrusions in its upper surface in both its compressed and expanded states, respectfully;

FIGS. 6A and 6B show an exemplary compressed substrate having a linear cut in its upper surface in both its compressed and expanded states, respectfully;

FIGS. 7A and 7B show an exemplary compressed substrate having two linear cuts in its upper surface in both its compressed and expanded states, respectfully;

FIGS. 8A and 8B show an exemplary compressed substrate having flowering pedals forming its upper surface in both its compressed and expanded states, respectfully; and

FIGS. 9A and 9B show an exemplary absorbent article including a compressed substrate in both its compressed and expanded states, respectfully.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to providing a compressed substrate within absorbent articles. The compressed substrate of the present invention does not substantially alter or interfere with the absorbent capabilities of the absorbent article by pressing against the absorbent core in the x- and y-directions. Thus, the compressed substrate can be included within conventional absorbent articles without significantly sacrificing the absorbency characteristics of the article.

The compressed substrate of the present invention is configured to expand toward the skin of the wearer (i.e., in the z-direction of the absorbent article perpendicular to the plane of the absorbent article) upon contact with a liquid. That is, the compressed substrate does not substantially expand in any direction parallel with the plane of the article (i.e., the x- and y-directions). As such, the compressed substrate does not significantly interfere with the absorbent capabilities of the absorbent article.

This z-directional expansion can be controlled by altering at least one surface of the compressed substrate. Altering the surface of the compressed substrate can allow for greater control over the direction and shape of the expanded compressed substrate upon contact with a liquid.

Additionally, by altering the surface of the compressed substrate, the resulting expanded compressed substrate can have increased amount of surface area. This increased amount of surface area allows for increased exposure of an active agent located on or within the compressed substrate. Thus, the active agent can more effectively perform its desired function through the increased amount of surface area exposing the active agent.

### I. Compressed Substrate

The compressed substrate is constructed from a highly compressed web material. After compression-molding of the web material, a compressed substrate is formed that is configured to expand only in the direction of the compression forces (i.e., only in the z-direction) upon wetting. Thus, the direction of expansion upon contact with a liquid can be predisposed, allowing the direction of expansion of the compressed substrate to be predetermined when included within an absorbent article.

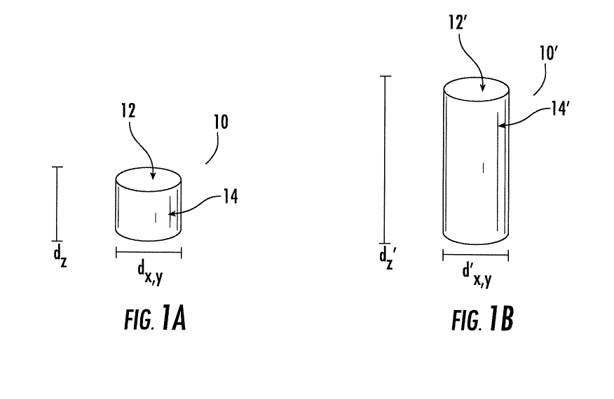

Referring to FIG. 1A, an exemplary compressed substrate 10 is shown in its dry, compressed state. The compressed substrate 10 has a compressed height d_{z} in its z-direction while still in its dry state. Upon contact with a liquid, the compressed substrate 10 expands to be an expanded compressed substrate 10′ having an expanded height d_{z}′ (as shown in FIG. 1B). The degree of expansion in the z-direction can be predetermined by the type of material included within the compressed substrate 10 and the force asserted in forming the compressed substrate 10.

The expansion of the compressed substrate 10 is substantially 1-dimensional. Upon contact with a liquid expansion of the compressed substrate 10 occurs in the z-direction, without substantially increasing the size of the compressed substrate 10 in either the x-direction or y-direction. For example, referring to FIGS. 1A and 1B, the compressed substrate 10 is shown having a cylindrical shape, such that its size in the x- and y-directions are substantially equal (i.e., the diameter of the cylindrical compressed substrate 10). The diameter d_{x,y} of the compressed substrate 10 remains substantially unchanged after contact with a liquid causing expansion in the z-direction. Thus, the diameter d_{x,y}′ of the expanded compressed substrate 10′ shown in FIG. 1B is nearly identical to the diameter d_{x,y} of the compressed substrate 10 shown in FIG. 1A (e.g., d_{x,y}′≦1.1 times d_{x,y}).

The expansion of the compressed substrate can be stated as an “expansion ratio” comparing of the degree of expansion in the z-direction compared to the degree of expansion in both the x- and y-directions (i.e., d_{z}′ divided by d_{z} compared to d_{x,y}′ divided by d_{x,y}). In particular embodiments, the compressed substrate can expand more than about 2:1.1 in the z-direction compared to the x- and y-directions, such as greater than 3:1.1, and from about 5:1.1 to about 10:1.1. For example, the expansion ration can be greater than about 2:1.05, such as greater than about 3:1.05, such as from about 5:1.05 to about 10:1.05.

For example, the compressed substrate 10 suitably expands to at least about 2 times its original height d_{z} in the z-direction when dry (i.e., expands 200%), and more suitably it expands to at least about 3 times the original height d_{z} when dry (i.e., expands 300%). For example, in some embodiments, the expanded compressed substrate 10′ can have a thickness or height d_{z}′ that is from about 5 times to about 10 times its original height d_{z} (i.e., expands from about 500% to about 1000%).

In one particular embodiment, the diameter d_{x,y}′ of the expanded compressed substrate 10′ can be less than about 110% of the diameter d_{x,y} of the compressed substrate 10 in a dry state (i.e., less than about 1.1 times the original diameter d_{x,y}), such as from 100% (i.e., unchanged in diameter upon contact with a liquid in the x- and y-directions) to about 107% (i.e., about 1.07 times the original diameter d_{x,y}). For instance, the diameter d_{x,y}′ of the expanded compressed substrate 10′ can be from about 100.5% to about 105% of the diameter d_{x,y} of the compressed substrate 10 in a dry state.

Of course, the compressed substrate 10 can be molded into any other shape, including but not limited to cuboids, cubes, cones, etc. No matter the particular shape of the compressed substrate 10, the dimensions in the x- and y-directions do not substantially increase upon contact with a liquid. Suitable compressed substrates are disclosed in U.S. patent application Ser. Nos. 11/955,916 and 11/955,937 filed on Dec. 13, 2007, the disclosures of which are incorporated in their entirety herein.

The compressed substrate 10 is configured to expand to the expanded compressed substrate 10′ nearly immediately upon contact with a small amount of a liquid. For example, the 1-dimensional expansion can occur within about 10 seconds of the compressed substrate 10 contacting a liquid, such expanding in less than about 5 seconds. In some embodiments, the 1-dimensional expansion of the compressed substrate 10 can occur from about 1 second to about 5 seconds, such as from about 1 second to about 3 seconds. Thus, the wearer of the absorbent article can be immediately alerted upon the first insult of the absorbent article.

In order to initiate the expansion of the compressed substrate 10, the compressed substrate 10 is configured to expand upon contact with a small amount of liquid. This amount of liquid need not completely saturate the compressed substrate 10. Of course, the amount of liquid necessary to cause complete expansion of the compressed substrate 10 to the expanded compressed substrate 10′ can vary with the size of the compressed substrate 10. However, when used in an absorbent article, the compressed substrate 10 is configured, in most embodiments, to expand upon contact with greater than about 0.1 milliliters (mL), such as from about 0.5 mL to about 15 mL, and from about 1 mL to about 12 mL. At these liquid levels, the compressed substrate 10 can at least double in height in the z-direction with an expansion ratio of at least 2:1.1, as stated above.

The compressed substrate offer the moisture triggered z-directional expansion with a significant amount of energy. Specifically, the compressed substrate can expand in the z-direction with an exerted force up to about 16 pounds per square inch (psi), such from about 10 psi to about 15 psi. Thus, the compressed substrate can press against the skin of the wearer with sufficient force to alert the wearer that an insult has occurred.

The web material that is compressed to form the compressed substrate can be a nonwoven web of fibers. Although the particular type of fiber is not a limitation of the invention, some fibers are particularly suitable for forming the compressed substrate 10 to be included within an absorbent article. The fibers may be, for example, any combination of synthetic or pulp fibers. The selected average fiber length and denier will generally depend on a variety of factors and desired processing steps.

In one embodiment, a substantial portion of the fibers may be cellulosic pulp staple fibers. Pulp fibers may be utilized to reduce costs, as well as impart other benefits to the compressed substrate 10, such as improved absorbency. Some examples of suitable cellulosic fiber sources include virgin wood fibers, such as thermomechanical, bleached and unbleached pulp fibers. Pulp fibers may have a high-average fiber length, a low-average fiber length, or mixtures of the same. Some examples of suitable high-average length pulp fibers include northern softwood, southern softwood, redwood, red cedar, hemlock, pine (e.g., southern pines), spruce (e.g., black spruce), combinations thereof, and so forth. Some examples of suitable low-average fiber length pulp fibers may include certain virgin hardwood pulps and secondary (i.e. recycled) fiber pulp from sources such as, for example, newsprint, reclaimed paperboard, and office waste. Hardwood fibers, such as eucalyptus, maple, birch, aspen, and so forth, may also be used as low-average length pulp fibers. These pulp fibers can be formed into a nonwoven web (e.g., a tissue web) according to any process (e.g., wetlaid, airlaid, bonded carded process, etc.).

In one particular embodiment, the web is a non-woven web of rayon material. In particular, the rayon material can be manufactured by a spun lace method in which a web is formed out of viscose rayon and fibers are coupled using a high-pressure water stream.

Alternatively, a majority of the fibers of the nonwoven web may be formed from synthetic polymers. Synthetic fibers can be formed into nonwoven fabrics or webs from many processes such as for example, meltblowing processes, spunbonding processes, bonded carded web processes, etc.

“Meltblown fibers” refers to fibers formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that may be continuous or discontinuous, are generally smaller than 10 microns in diameter, and are generally tacky when deposited onto a collecting surface.

“Spunbonded fibers” refers to small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Petersen, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers can sometimes have diameters less than about 40 microns, and are often between about 5 to about 20 microns.

Exemplary synthetic polymers for use in forming nonwoven web may include, for instance, polyolefins, e.g., polyethylene, polypropylene, polybutylene, etc.; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; and so forth. If desired such as those described above, may also be employed. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven web. Monocomponent fibers are generally formed from a polymer or blend of polymers extruded from a single extruder. Multicomponent fibers are generally formed from two or more polymers (e.g., bicomponent fibers) extruded from separate extruders. The polymers may be arranged in substantially constantly positioned distinct zones across the cross-section of the fibers. The components may be arranged in any desired configuration, such as sheath-core, side-by-side, pie, island-in-the-sea, three island, bull's eye, or various other arrangements known in the art. Various methods for forming multicomponent fibers are described in U.S. Pat. No. 4,789,592 to Taniguchi et al. and U.S. Pat. No. 5,336,552 to Strack, et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference thereto for all purposes. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference thereto for all purposes.

Although any combination of polymers may be used, the polymers of the multicomponent fibers are typically made from thermoplastic materials with different glass transition or melting temperatures where a first component (e.g., sheath) melts at a temperature lower than a second component (e.g., core). Softening or melting of the first polymer component of the multicomponent fiber allows the multicomponent fibers to form a tacky skeletal structure, which upon cooling, stabilizes the fibrous structure. For example, the multicomponent fibers may have from about 5% to about 80%, and in some embodiments, from about 10% to about 60% by weight of the low melting polymer. Further, the multicomponent fibers may have from about 95% to about 20%, and in some embodiments, from about 90% to about 40%, by weight of the high melting polymer. Some examples of known sheath-core bicomponent fibers available from KoSa Inc. of Charlotte, N.C. under the designations T-255 and T-256, both of which use a polyolefin sheath, or T-254, which has a low melt co-polyester sheath. Still other known bicomponent fibers that may be used include those available from the Chisso Corporation of Moriyama, Japan or Fibervisions LLC of Wilmington, Del.

Fibers of any desired length may be employed, such as staple fibers, continuous fibers, etc. In one particular embodiment, for example, staple fibers may be used that have a fiber length in the range of from about 1 to about 150 millimeters, in some embodiments from about 5 to about 50 millimeters, in some embodiments from about 10 to about 40 millimeters, and in some embodiments, from about 10 to about 25 millimeters. Although not required, carding techniques may be employed to form fibrous layers with staple fibers as is well known in the art. For example, fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be bonded using known techniques to form a bonded carded nonwoven web.

If desired, the nonwoven web may have a multi-layer structure. The other layers can be other nonwoven webs, films, and the like. For example, in one embodiment, at least two nonwoven webs can be combined to form a nonwoven laminate. Suitable multi-layered materials may include, for instance, spunbond/meltblown/spunbond (SMS) laminates and spunbond/meltblown (SM) laminates. Various examples of suitable SMS laminates are described in U.S. Pat. No. 4,041,203 to Brock et al.; U.S. Pat. No. 5,213,881 to Timmons, et al.; U.S. Pat. No. 5,464,688 to Timmons, et al.; U.S. Pat. No. 4,374,888 to Bornslaeger; U.S. Pat. No. 5,169,706 to Collier, et al.; and U.S. Pat. No. 4,766,029 to Brock et al., which are incorporated herein in their entirety by reference thereto for all purposes. In addition, commercially available SMS laminates may be obtained from Kimberly-Clark Corporation under the designations Spunguard® and Evolution®.

Another example of a multi-layered structure is a spunbond web produced on a multiple spin bank machine in which a spin bank deposits fibers over a layer of fibers deposited from a previous spin bank. Such an individual spunbond nonwoven web may also be thought of as a multi-layered structure. In this situation, the various layers of deposited fibers in the nonwoven web may be the same, or they may be different in basis weight and/or in terms of the composition, type, size, level of crimp, and/or shape of the fibers produced. As another example, a single nonwoven web may be provided as two or more individually produced layers of a spunbond web, a carded web, etc., which have been bonded together to form the nonwoven web. These individually produced layers may differ in terms of production method, basis weight, composition, and fibers as discussed above.

A nonwoven web constructed from synthetic fibers may also contain an additional fibrous component such that it is considered a composite. For example, a nonwoven web may be entangled with another fibrous component using any of a variety of entanglement techniques known in the art (e.g., hydraulic, air, mechanical, etc.). In one embodiment, the nonwoven web is integrally entangled with cellulosic fibers using hydraulic entanglement. Hydraulically entangled nonwoven webs of staple length and continuous fibers are disclosed, for example, in U.S. Pat. No. 3,494,821 to Evans and U.S. Pat. No. 4,144,370 to Boulton, which are incorporated herein in their entirety by reference thereto for all purposes. Hydraulically entangled composite nonwoven webs of a continuous fiber nonwoven web and a pulp layer are disclosed, for example, in U.S. Pat. No. 5,284,703 to Everhart, et al. and U.S. Pat. No. 6,315,864 to Anderson, et al., which are incorporated herein in their entirety by reference thereto for all purposes.

No matter the particular construction of the nonwoven web, the web is compression molded into a compressed substrate 10 configured to expand 1-dimensionally. The 1-dimensional expansion generally occurs in the direction of the compression forces exerted during the formation of the compressed substrate 10. Thus, one of ordinary skill in the art would be able to form a compressed substrate 10 having any desired shape and any desired expansion parameters.

In one embodiment, the compressed web materials can be formed by first folding or rolling the web material into a tube-like shape, such that the web material is generally longer in the z-direction than in the x- and y-directions. This folded or rolled web material is then placed into an elongated barrel such that the longer z-direction of the folded or rolled web is parallel with the length of the barrel. The shape of the barrel in the x- and y-directions corresponds to the shape of the resulting compressed substrate 10. For example, to make the compressed substrate 10 shown in FIG. 1A, the barrel shape is cyclical such that the x- and y-directions of the barrel define a circle (or oval). Alternatively, the barrel shape can define any desired shape in the x- and y-directions to produce the compressed substrate 10 in the desired shape.

After placement in the barrel, the folded or rolled web is subjected to a compression force in a direction of the elongation of the barrel (i.e., the z-direction). This compression force is sufficient to compress the folded or rolled web into a compressed substrate 10 that will not retain its initial shape until after exposure to a liquid. That is, the disposable tissue should be subjected to compression molding under a pressure within a predetermined pressure range that varies according to the shape, configuration, and chemical construction of the web as described above. However, if the web is pressed under a pressure within the predetermined pressure range, it is compressed at a compressibility (ΔV/V) in a range of 0.4 to 0.6. Here, the compressibility (ΔV/V) represents a ratio of the amount of volume change (ΔV) in the compressed substrate 10 to the volume (V) of the uncompressed web. The amount of volume change means the difference between the volume (V) of the uncompressed web and the volume of the compressed substrate 10.

For example, when making a compressed substrate 10 shaped as in FIG. 1A with a diameter d_{x,y} of about 2 cm and a height d_{z} of about 1 cm from a web. The web can have any initial size, such as less than about 20 cm×20 cm, such as from about 5 cm×5 cm to about 15 cm×15 cm. In one particular embodiment, the web can have an initial size of about 10×10 cm. The compression force can be apply a pressure to the folded or rolled tissue web of about 95 kiloNewton (kN) to about 300 kN, such as from about 145 kN to about 250 kN. In one particular embodiment the compression force can be from about 190 kN to about 200 kN in the z-direction.

Although the apparatus for forming the compressed substrate 10 can vary, a particularly suitable apparatus can include a cylindrical molding barrel having a longitudinal, through passage. The molding barrel can be supported on a table such that both end portions of the through passage of the molding barrel are exposed to the outside. An upper press can be installed vertically movably above the table and having a pressing rod to be inserted into the through passage of the molding barrel when the upper press moves downwardly. A lower press can also be installed vertically movably below the table and having a supporting rod to be inserted into the through passage of the molding barrel when the lower press moves upwardly.

In this set up, the upper press can include a power source for pressing the folded or rolled web received in the through passage. The supporting rod of the lower press closes an entrance of the through passage of the molding barrel to compression-mold the folded or rolled web and opens the entrance of the through passage to discharge the compressed substrate 10 from the through passage. The compressed substrate 10 is molded to have a shape that is the same as a space defined by the through passage of the molding barrel, the supporting rod of the lower press, and the pressing rod of the upper press. In a state where the entrance of the through passage of the molding barrel is opened, the compressed substrate 10 is discharged from the through passage by the upper press moving downwardly.

In one particular embodiment, the compressed web materials can be made with the compression molding apparatus and methods described in International Publication No. WO 200/082448 A1 of Lee, et al., the disclosure of which is incorporated herein by reference.

### II. Increasing the Surface Area of the Compressed Substrate

According to the present invention, at least one surface of the compressed substrate can be altered to control the expansion of the substrate and provide an increased amount of surface area of the upper surface. As the surface area of the upper surface increases, any active agent within the compressed substrate or on the upper surface of the compressed substrate becomes more exposed to the outer environment. Thus, the active agent can be more efficiently transferred out of the compressed substrate, and the purpose of the active agent can be more readily achieved.

The surface area of the altered upper surface of the compressed substrate can increase from an initial surface area to an expanded surface area by a factor of at least about 1.1 (e.g., at least about 110% of the initial surface area), such as greater than about 1.25 (e.g., at least about 125% of the initial surface area). For example, the surface area of the altered upper surface of the compressed substrate can increase from its initial surface area to its expanded surface area by a factor of at least about 2 (e.g., at least about 200% of the initial surface area), such as greater than about 3 (e.g., at least about 300% of the initial surface area).

A. Concave Upper Surface

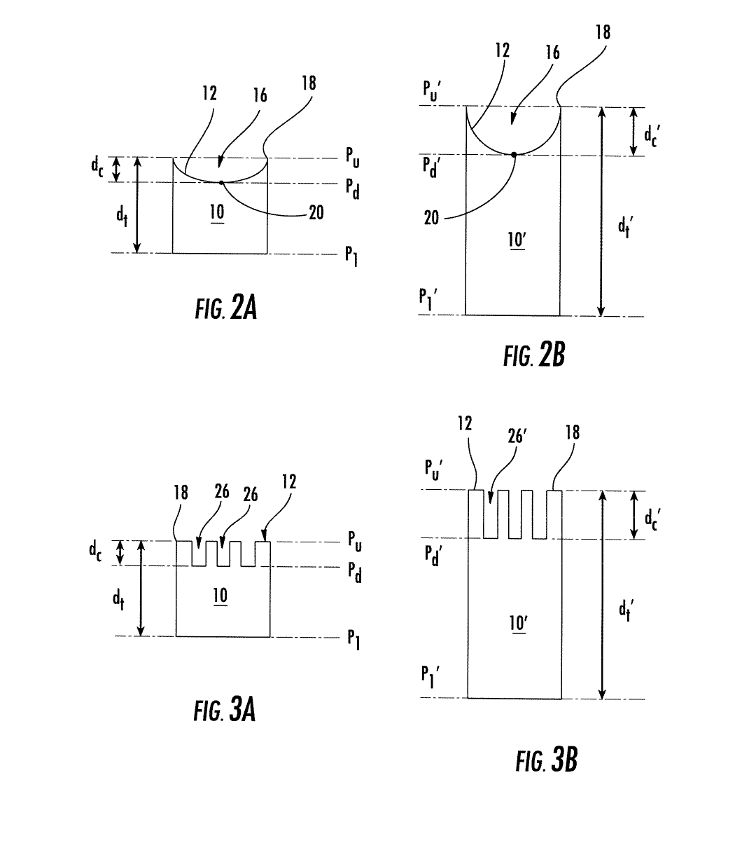

In one embodiment, the upper surface 11 of the compressed substrate 10 can have a shaped surface. In one embodiment, the upper surface can be modified to be concave in shape. Referring to FIG. 2A, a compressed substrate 10 is shown having an upper surface 12 that is concave in shape. As shown, the concave upper surface 12 curves inwardly, toward the center of the compressed substrate 10. The concave upper surface 12 forms a cavity 16 depressed into the compressed substrate 10 and is defined by the outer, upper edges 18. The curvature of the concave upper surface can vary, depending on the desired size of the cavity 16 to be formed in the upper surface 12 of the compressed substrate 10.

The depth d_{c} of the cavity 16 of the can be measured as the distance in the z-direction from the upper edge 18 to the most depressed point 20 in the concave upper surface 12. To measure this distance in the z-direction, the distance between two imaginary, parallel planes is measured. Both planes are defined by the x,y plane that is perpendicular to the z-direction. First, the upper plane Pᵤ is defined by the plane in the x,y-directions that includes the upper most point(s) of the upper edge 18. Second, the depressed plane P_{d} is defined by the plane in the x,y-directions that includes the lower, most depressed point within the cavity 16 of the upper surface 12. The depth d_{c} of the cavity 16 is distance between these two planes.

The depth d_{c} of the cavity 16 can vary as desired. In many embodiments, the depth d_{c} of the cavity 16 varies according to the size of the compressed substrate. Thus, the depth d_{c} of the cavity 16 can also be expressed as a percentage of the entire thickness dₜ in the z-direction of the compressed substrate 10. The thickness dₜ in the z-direction of the compressed substrate 10 is measured from the upper plane Pᵤ to the lower plane Pₗ that includes the lowest point(s) of the compressed substrate 10. Thus, the percentage of the depth d_{c} compared to the thickness dₜ of the compressed substrate can be calculated according to the formula:

d_{c}/dₜ×100=depth percentage

In one embodiment, the depth percentage of the cavity 16 can be at least about 5%, such as from about 10% to about 50%. In some particular embodiments, the depth percentage of the cavity can be from about 15% to about 30%. For example, when the compressed substrate has a thickness dₜ of about 1 centimeter (10 millimeters), the depth of the cavity can be at least about 0.5 millimeters, such as from about 1 millimeter to about 5 millimeters.

When the compressed substrate 10 expands to its expanded state upon contact with a liquid, the depth d_{c} of the cavity 16 can also increase. Referring to FIG. 2B, the expanded substrate 10′ is shown having an expanded depth d_{c}′ that is greater than the original depth d_{c} of the compressed substrate 10. The expanded depth d_{c}′ can be at least about 1.5 times its original depth d_{c} in the z-direction when dry (i.e., expands 50%), and more suitably it expands to at least about 2 times the original depth d_{c} when dry (i.e., expands 200%).

The upper surface 12 of the compressed substrate 10 can be formed with a concave shape by shaping the compressed substrate 10 after its formation. In this embodiment, the compressed substrate 10 can be formed first, and then a cavity 16 can be cut (e.g., drilled) into the upper surface 14 of the compressed substrate 10. Alternatively, the upper surface 12 of the compressed substrate 10 can be formed with a concave shape during the formation of the compressed substrate 10. During compression, the pressing rod used to compress the material can have a convex contact surface, so as to form a compressed substrate 10 having a concave upper surface 12.

B. Plurality of Apertures in Upper Surface

Instead of having a single cavity 16 in the upper surface 12, the compressed substrate 10 can, in another embodiment, define a plurality of apertures 22 in the upper surface 12, as shown in FIGS. 3A and 3B. The number of apertures 22 present on the upper surface 12 can vary according to the size of the available surface area defined by the upper surface 12. In one embodiment, the upper surface 12 can define at least about two apertures 22, such as at least two apertures 22 per centimeter squared (cm²) of the surface area defined by the upper surface 12, such as at least about three apertures 26 per cm².

The shape and depth of each aperture 22 can also vary as desired. The depth dₐ of the apertures 22 is measured as the distance in the z-direction from the upper edge 18 to the most depressed point 24 in the aperture 26. The depth dₐ of the apertures 22 can be, as with the cavity 16 in the compressed substrates shown in FIGS. 2A and 2B, expressed as a percentage of the entire thickness dₜ in the z-direction of the compressed substrate 10. In one embodiment, the average depth percentage of the apertures 22 can be at least about 5%, such as from about 10% to about 50%. In some particular embodiments, the average depth percentage of the apertures 22 can be from about 15% to about 30%. For example, when the compressed substrate has a thickness dₜ of about 1 centimeter (10 millimeters), the average depth dₐ of the apertures 22 can be at least about 0.5 millimeters, such as from about 1 millimeter to about 5 millimeters.

When the compressed substrate 10 expands to its expanded state upon contact with a liquid, the average depth dₐ of apertures 22 can also increase. Referring to FIG. 3B, the expanded substrate 10′ is shown having an expanded depth dₐ′ that is greater than the original depth dₐ of the compressed substrate 10. The expanded depth dₐ′ can be at least about 1.5 times its original depth dₐ in the z-direction when dry (i.e., expands 50%), and more suitably it expands to at least about 2 times the original depth d_{c} when dry (i.e., expands 200%).

The upper surface 12 of the compressed substrate 10 can be formed with a plurality of apertures 22 by shaping the compressed substrate 10 after its formation. In this embodiment, the compressed substrate 10 can be formed first, and then a plurality of apertures 22 can be cut (e.g., drilled) into the upper surface 14 of the compressed substrate 10. Alternatively, the upper surface 12 of the compressed substrate 10 can be formed with a plurality of apertures 22 during the formation of the compressed substrate 10. During compression, the pressing rod used to compress the material can have a plurality of projections on the contact surface. Upon compression, the compressed substrate 10 has a plurality of apertures 22 that correspond to the size and shape of the projections on the contact surface.

C. Convex Upper Surface

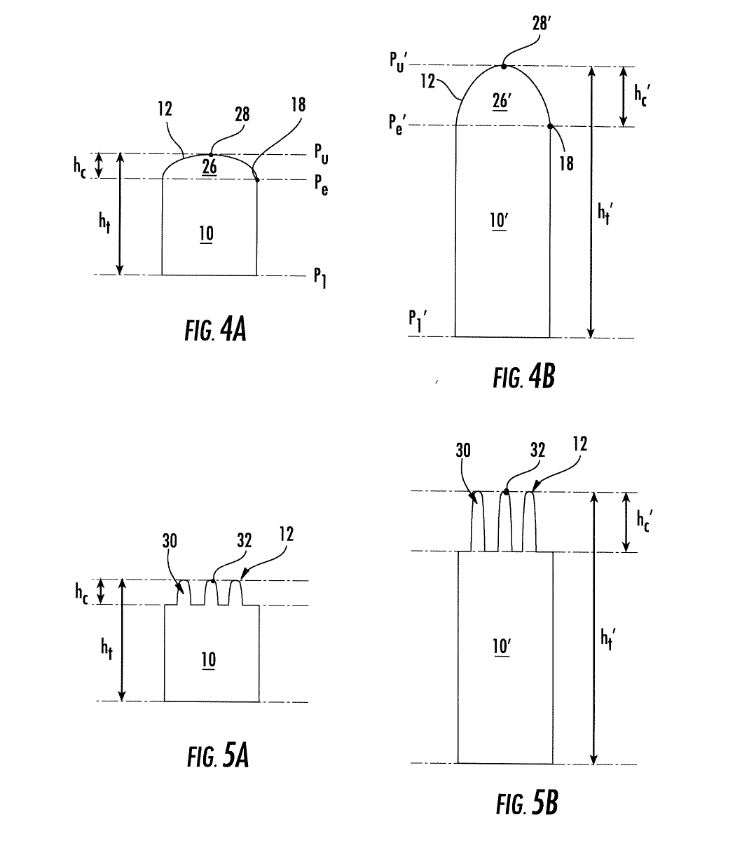

In another embodiment, the compressed substrate 10 can have an upper surface 12 that has a convex shape. Referring to FIGS. 4A and 4B, a compressed substrate 10 is shown having an upper surface 12 that is convex in shape. As shown, the convex upper surface 12 curves outwardly, away from the center of the compressed substrate 10. The curvature of the convex upper surface can vary, depending on the desired size of the cone 26 of the upper surface 12 of the compressed substrate 10.

The height h_{c} of the cavity 22 of the can be measured as the distance in the z-direction from the upper edge 18 to the outermost point 28 on the cone 26. To measure this distance in the z-direction, the distance between two imaginary, parallel planes is measured. Both planes are defined by the x,y plane that is perpendicular to the z-direction. First, the upper plane Pᵤ is defined by the plane in the x,y-directions that includes the outermost point 28 of the cone 26. Second, the edge plane Pₑ is defined by the plane in the x,y-directions that includes the upper edge 18 of the side surface 14 of the upper surface 12. The height h_{c} of the cone 26 is distance between these two planes.

The height h_{c} of the cone 26 can vary as desired. In many embodiments, the height h_{c} of the cone 26 varies according to the size of the compressed substrate. Thus, the height h_{c} of the cone 26 can also be expressed as a percentage of the side thickness dₜ in the z-direction of the compressed substrate 10. The side thickness dₜ in the z-direction of the compressed substrate 10 is measured from the edge plane Pᵤ to the lower plane Pₗ that includes the lowest point(s) of the compressed substrate 10. Thus, the percentage of the height h_{c} of the cone 26 compared to the side thickness dₜ of the compressed substrate can be calculated according to the formula:

h_{c}/hₜ×100=height percentage

In one embodiment, the height percentage of the cone 26 can be at least about 5%, such as from about 10% to about 50%. In some particular embodiments, the height percentage of the cone 26 can be from about 15% to about 30%. For example, when the compressed substrate has a side edge thickness dₜ of about 1 centimeter (10 millimeters), the height of the cone can be at least about 0.5 millimeters, such as from about 1 millimeter to about 5 millimeters.

When the compressed substrate 10 expands to its expanded state upon contact with a liquid, the height h_{c} of the cone 26 also increases. Referring to FIG. 4B, the expanded substrate 10′ is shown having an expanded cone height h_{c}′ that is greater than the original height h_{c} of the cone 26 of the compressed substrate 10. The expanded height h_{c}′ of the cone 26 can be at least about 1.5 times its original height h_{c} in the z-direction when dry (i.e., expands 50%), and more suitably it expands to at least about 2 times the original height h_{c} when dry (i.e., expands 200%).

The upper surface 12 of the compressed substrate 10 can be formed with a convex shape by shaping the compressed substrate 10 after its formation. In this embodiment, the compressed substrate 10 can be formed first, and then a cone 26 can be shaped (e.g., cut) into the upper surface 14 of the compressed substrate 10. Alternatively, the upper surface 12 of the compressed substrate 10 can be formed with a convex shape during the formation of the compressed substrate 10. During compression, the pressing rod used to compress the material can have a concave contact surface, so as to form a compressed substrate 10 having a convex upper surface 12.

D. Plurality of Protuberances in the Upper Surface

Instead of having a single cone 26 in the upper surface 12, the compressed substrate 10 can, in another embodiment, define a plurality of protuberances 30 in the upper surface 12, as shown in FIGS. 5A and 5B. The number of protuberances 30 present on the upper surface 12 can vary according to the size of the available surface area defined by the upper surface 12. In one embodiment, the upper surface 12 can define at least about two protuberances 30, such as at least two protuberances 30 per centimeter squared (cm²) of the surface area defined by the upper surface 12, such as at least about three protuberances 30 per cm².

The shape and height of each protuberance 30 can vary as desired. The height hₚ of the protuberances 30 is measured as the distance in the z-direction from the upper side edge 18 to the outermost point 32 on the protuberance 30. The height hₚ of the protuberances 30 can be, as with the cone 26 in the compressed substrates shown in FIGS. 4A and 4B, expressed as a percentage of the side edge height hₜ in the z-direction of the compressed substrate 10. In one embodiment, the average height percentage of the protuberances 30 can be at least about 5%, such as from about 10% to about 50%. In some particular embodiments, the average height percentage of the protuberances 30 can be from about 15% to about 30%. For example, when the compressed substrate has a side-edge height ht of about 1 centimeter (10 millimeters), the average height hₚ of the protuberances 30 can be at least about 0.5 millimeters, such as from about 1 millimeter to about 5 millimeters.

When the compressed substrate 10 expands to its expanded state upon contact with a liquid, the average height hₚ of protuberances 30 can also increase. Referring to FIG. 5B, the expanded substrate 10′ is shown having an expanded height hₚ′ that is greater than the original height hₚ of the compressed substrate 10. The expanded height hₚ′ can be at least about 1.5 times its original height hₚ in the z-direction when dry (i.e., expands 50%), and more suitably it expands to at least about 2 times the original height hₚ when dry (i.e., expands 200%).

The upper surface 12 of the compressed substrate 10 can be formed with a plurality of protuberances 30 by shaping the compressed substrate 10 after its formation. In this embodiment, the compressed substrate 10 can be formed first, and then a plurality of protuberances 30 can be cut into the upper surface 14 of the compressed substrate 10 by removing the areas around the protuberances 30. Alternatively, the upper surface 12 of the compressed substrate 10 can be formed with a plurality of protuberances 30 during the formation of the compressed substrate 10. During compression, the pressing rod used to compress the material can have a plurality of apertures on the contact surface. Upon compression, the compressed substrate 10 has a plurality of protuberances 30 that correspond to the size and shape of the apertures on the contact surface.

E. Linear Cuts in the Upper Surface

The upper surface 12 of the compressed substrate 10 can include, in one embodiment, at least one linear cut. In addition to increasing the surface area of the upper surface upon expansion, linear cuts in the upper surface provide a unique ability to control the direction of expansion of the upper portion of the compressed substrate 10 upon contact with liquid. As explained above, the expansion of the compressed substrate 10 is generally limited to the z-direction. However, the present inventors have surprisingly found that linear cuts in the upper surface 12 of the compressed substrate 10 allows for the upper portion to have a direction of expansion in the z-direction with a component in the x,y-directions. Specifically, upon wetting, the upper portion expands in the z-direction, but also at an angle away from a center axis in the z-direction.

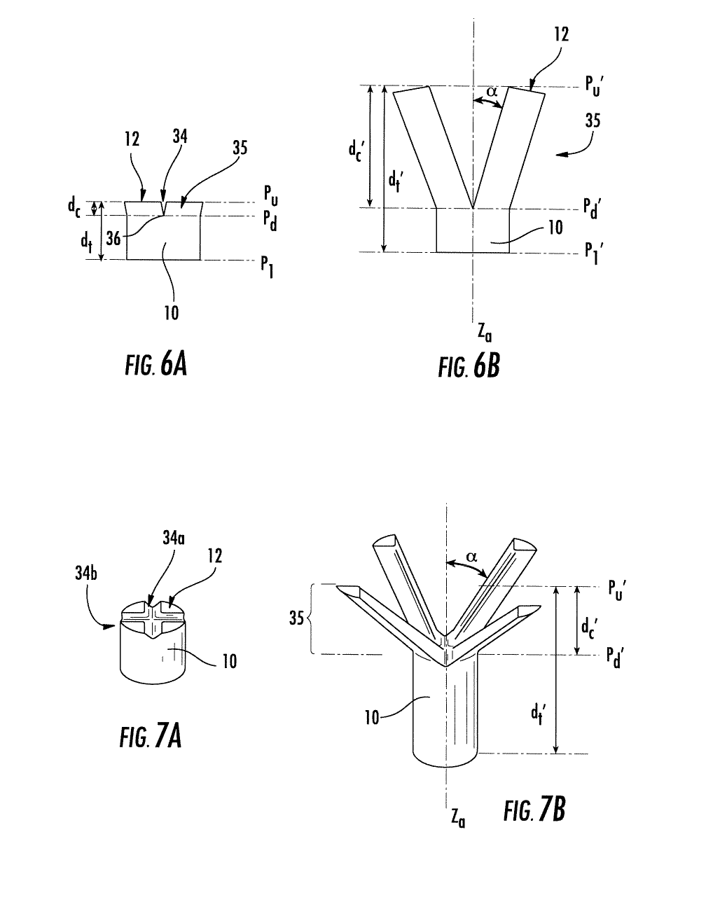

FIG. 6A shows a compressed substrate 10 having a single linear cut 34 in its upper surface 12. When expanded, upon contact with a liquid, the upper portion 35 (defined by the portion of the compressed substrate 10 encompassed within the depth d_{lc} of the linear cut 34 and above the bottom point 35) expands at an acute angle α away from the center axis Zₐ in the z-direction. This acute angle α is, by definition, less than 90°, and is preferably less than 45° such that a majority of the expansion is in the z-direction. For example, the acute angle α can be from about 1° to about 40°, such as from about 5° to about 30°, or from about 10° to about 25°.

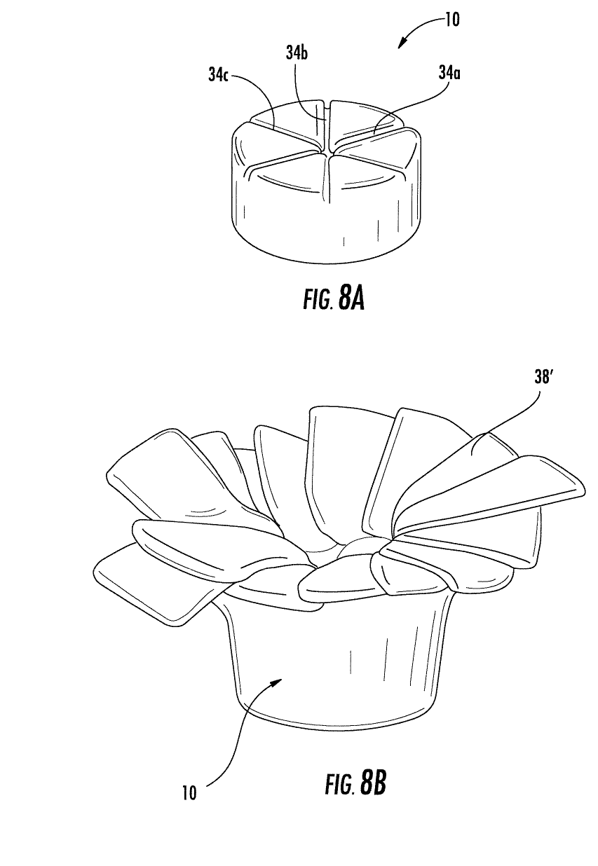

In other embodiments, more than one linear cut 34 can be included in the upper surface 12. For example, FIG. 7A shows a compressed substrate 10 having two linear cuts 34a, 34b in its upper surface 12. The two linear cuts 34a, 34b are shown forming a cross-like shape in the upper surface 12. However, any number of liner cuts 34 can be used.

The depth of the linear cut(s) 34 can vary as desired. The depth d_{lc} of the linear cut 34 can vary as desired. In many embodiments, the depth d_{lc} of the linear cut 34 varies according to the size of the compressed substrate. Thus, depth d_{lc} of the linear cut 34 can also be expressed as a percentage of the entire thickness dₜ in the z-direction of the compressed substrate 10. The thickness dₜ in the z-direction of the compressed substrate 10 is measured from the upper plane Pᵤ defined by the upper surface 12 to the lower plane Pₗ that includes the lowest point(s) of the compressed substrate 10.

In one embodiment, the depth percentage of the linear cut 34 can be at least about 5%, such as from about 10% to about 50%. In some particular embodiments, the depth percentage of the linear cut 34 can be from about 15% to about 30%. For example, when the compressed substrate has a thickness dₜ of about 1 centimeter (10 millimeters), the depth of the linear cut 34 can be at least about 0.5 millimeters, such as from about 1 millimeter to about 5 millimeters.

The linear cut(s) 34 can be cut into the upper surface 12 of the compressed substrate after its formation by any method (e.g., using a blade, laser, etc.). Alternatively, the linear cut(s) 34 can be formed in the upper surface 12 during compression through the use of a pressing rod that has a linear protuberance(s) in its contact surface. Thus, upon compression, the upper surface of the compressed substrate has a linear cut(s) 34 that correspond to the linear protuberance(s) on the pressing rod.

F. Flowering Expansion of Upper Surface

The upper surface 12 of the compressed substrate 10 can be formed such that, upon contact with a liquid, it “blossoms” into an expanded compressed substrate 10′ having pedals 38, as shown in FIGS. 8A and 8B. In one particular embodiment, at least three linear cuts 34a-34c extending across the upper surface through the center of the upper surface of the compressed substrate 10 are substantially equally spaced apart, as shown in FIG. 8A. These linear cuts are substantially uniform in depth. For example, the depth of each cut can be from about 75% to about 25%, such as from about 60% to about 40%. Thus, the linear cuts extend approximately to the center of the compressed substrate 10.

Upon contact with a liquid, the expansion of the compressed substrate occurs in the z-direction for the uncut portion; however, the cut upper portion blossoms from the center. Effectively, the individual layers of the web material in the center of the cut upper section separate from each other to form an expanded substrate similar to a blooming flower, where the individual layers of the web material form “pedals” of the flower that open from the inside of the compressed substrate. When cut like a pie (as shown in FIG. 8A), the individual web layers are basically free to separate and blossom in the center region of the upper portion of the compressed substrate, while the layers of web material along the edges are still compressed together. Thus, the center layers separate and blossom open, while the edge layers remain in contact with each other.

This flowering expansion of the upper portion of the compressed substrate dramatically increases the surface area of the compressed substrate. For example, the surface area of the upper portion of the expanded, bloomed compressed substrate can increase from its initial surface area to its expanded surface area by a factor of at least about 2 (e.g., at least about 200% of the initial surface area), such as greater than about 3 (e.g., at least about 300% of the initial surface area). In some particular embodiments, the surface area of the upper portion of the expanded, bloomed compressed substrate can increase from its initial surface area to its expanded surface area by a factor of at least about 5 (e.g., at least about 500% of the initial surface area), such as greater than about 10 (e.g., at least about 1,000% of the initial surface area).

### III. Active Agents

No matter its shape, the increased surface area of the upper surface 12 of allows for greater exposure of an active agent within the compressed substrate 10 to the outer environment, especially when expanded. Thus, the desired effect of the active agent can be more efficiently accomplished. When expanded, the surface area of the upper surface 12 of the compressed substrate 10 increases even more, allowing greater exposure of any active agent in the compressed substrate.

Additionally, the active agent can be hidden within the construction of the compressed substrate and rendered effectively inactive since it is substantially concealed from the outer environment. However, upon expansion, the wicking action of the liquid can transport the active agent to the surfaces, especially to the upper surface, to be exposed to the outer environment and the benefit of the active agent can be realized. As such, in one particular embodiment, the active agent can be applied only to the internal portions of the compressed substrate. For example, the active agent can be applied to the web material only to a particular portion (e.g., the center, along one edge, etc.), then the web material can be folded, rolled, and/or placed into the compression apparatus such that the active agent is presently only in the center and/or internal portions of the compressed substrate.

The web material that is compressed to form the compressed substrate 10 can be applied to (e.g., sprayed on, printed on, saturated with, etc.) the active agent. In one embodiment, the active agent is applied to the entire web material. Alternatively, the active agent can be applied only the area of the web material that forms the upper portion, including the upper surface 12, of the compressed substrate 10 that is subsequently formed. In yet another embodiment, an active agent can be applied directly to the upper surface 12 of the compressed substrate 10. Of course, in this embodiment, the active agent must be applied in a non-liquid form to avoid expansion of the compressed substrate 10.

The active agent can indicate that the compressed substrate has been wetted, especially when the compressed substrate is included within an absorbent article. In one embodiment, neurosensory agents (agents that induce a perception of temperature change without involving an actual change in temperature such as, for example peppermint oil, eucalyptol, eucalyptus oil, methyl salicylate, camphor, tea tree oil, ketals, carboxamides, cyclohexanol derivatives, cyclohexyl derivatives, and combinations thereof) can be included in the compressed substrate. For example, a neurosensory agent can be included on or in the compressed substrate 10 to provide a cue in the form of a cooling sensation to the skin of the wearer upon contact. Thus, when the compressed substrate 10 expands upon contact with a liquid, the expanded compressed substrate 10′ pressed to the skin of the wearer can create a cooling sensation, further alerting the wearer that an insult of the absorbent article has occurred.

The physiological cooling agent can be, in one embodiment, a polyol. Many polyols are known to provide a cooling sensation upon contact with skin due to their endothermic (heat-absorbing) reaction when dissolving in moisture (e.g., the liquid insulting the absorbent article, the moisture located on the skin, etc.). Suitable polyols can include those of a hydrogenated form of carbohydrate, whose carbonyl group (aldehyde or ketone, reducing sugar) has been reduced to a primary or secondary hydroxyl group. These polyols can have a general formula H(HCOH)ₙ₊₁H, whereas sugar's is H(HCOH)ₙHCO, where n is an integer from 0 to 10. Exemplary polyols can include, but are not limited to, glycol, glycerol, erythritol, arabitol, xylitol, zylitol, mannitol, sorbitol, and the like. The use of such a physiological cooling agent can provide a wetness sensation on the skin of the wearer without actual moisture remaining on the skin.

Other suitable cooling agents are chemical compounds that have a negative heat of solution; that is, suitable cooling agents are chemical compounds that when dissolved in water feel cool due to an endothermic chemical reaction. Some suitable cooling agents for inclusion in the compressed substrate include, for example, ammonium nitrate, sodium chloride, potassium chloride, xylitol, barium hydroxide, barium oxide, magnesium potassium sulfate, potassium aluminum sulfate, sodium borate, sodium phosphate, and combinations thereof. Similar to the heating agents described herein, in some embodiments, the cooling agent may be surrounded by a hydrophobic wax material prior to being incorporated into the matrix material.

In yet another tactile cue, a tingling sensation can be created on the skin of the wearer upon contact with a liquid. The tingling sensation is generated from gas formation, such as disclosed in U.S. Pat. No. 6,929,819 of Underhill, et al., which is incorporated herein. Generally, the compressed substrate can include an effervescent agent or combination of agents that alerts the wearer that urination has occurred by releasing gas and causing a mild concussive (i.e., “popping,” “crackling,” “bubbling” or “fizzing”) sensation on or next to the wearer's skin upon urination. This may be accomplished without trapping moisture against the skin of the wearer. One example of a suitable acid/base combination is shown in equation (1)

NaHCO₃+KHC₄H₄O₆→KNaC₄H₄O₆+H₂O+CO₂ (1)

In equation (1), sodium bicarbonate and potassium bitartrate react in the presence of a liquid (urine) to form carbon dioxide gas and by-products. The production of the carbon dioxide gas alerts the wearer of the pad containing the acid and base that urination has occurred.

Another suitable acid/base combination is shown in equation (2):

NaAl(SO₄)₂+3NaHCO₃→Al(OH)₃+2Na₂SO₄+3CO₂ (2)

In equation (2), sodium aluminum sulfate and sodium bicarbonate react in the presence of liquid (urine) to form carbon dioxide gas and by-products. Other acids that can be used in combination with sodium bicarbonate to produce an effervescent agent in accordance with the present invention include ascorbic, lactic, glycolic, malic, tartaric, and fumaric. When mixed with sodium bicarbonate and contacted with urine, these acids produce carbon dioxide gas. This gas production can alert the wearer that urination has occurred.

In another embodiment, the active agent can deliver a beneficial affect to the skin of the user or wearer. For example, the active agent can be an antimicrobial agent, moisturizer, nutrients (e.g., anti-oxidants, transdermal drug delivery agents, botanical extracts, vitamins, magnets, magnetic metals, foods, and drugs), surface conditioning agents (e.g., pH adjusting agents, moisturizers, skin conditioners, exfoliation agents, lubricants, skin-firming agents, anti-callous agents, anti-acne agents, anti-aging agents, anti-wrinkle agents, anti-dandruff agents, wound care agents, skin lipids, enzymes, scar care agents, humectants, powders, botanical extracts, and drugs), anti-inflammatory agents (e.g., health ingredients, skin conditioners, external analgesic agents, anti-irritant agents, anti-allergy agents, anti-inflammatory agents, wound care agents, transdermal drug delivery, and drugs), emotional benefit agents (e.g., fragrances, odor neutralizing materials, exfoliation agents, skin-firming agents, anti-callous agents, anti-acne agents, anti-aging agents, soothing agents, calming agents, external analgesic agents, anti-wrinkle agents, anti-dandruff agents, antiperspirants, deodorants, wound care agents, scar care agents, coloring agents, powders, botanical extracts and drugs), etc.

In yet another embodiment, the active agent can be an odor adsorber, such as activated carbon. The activated carbon can be in particle form or can be bonded to the web material. Generally speaking, activated carbon may be formed from a variety of sources, such as from sawdust, wood, charcoal, peat, lignite, bituminous coal, coconut shells, etc. Some suitable forms of activated carbon and techniques for formation thereof are described in U.S. Pat. No. 5,693,385 to Parks; U.S. Pat. No. 5,834,114 to Economy. et al.; U.S. Pat. No. 6,517,906 to Economy, et al.; U.S. Pat. No. 6,573,212 to McCrae, et al., as well as U.S. Patent Application Publication Nos. 2002/0141961 to Falat. et al. and 2004/0166248 to Hu. et al., all of which are incorporated herein in their entirety by reference. Regardless, the concentration of activated carbon is generally tailored to facilitate odor control without adversely affecting other properties of the compressed substrate. For instance, activated carbon may be present in the compressed substrate in an amount from about 1 wt. % to about 50 wt. %, in some embodiments from about 2 wt. % to about 30 wt. %, and in some embodiments, from about 5 wt. % to about 20 wt. %.

An olfactory cue can also be incorporated into the compressed substrate. When dry, the olfactory cue is minimized during dry wear. However, upon wetting, the olfactory cue is released. This olfactory can alert a care giver of the wearer that wetting has occurred (e.g., an insult of the absorbent article has occurred). Additionally, the olfactory cue can disguise any scent released by the bodily fluids contained within the absorbent article upon insult (e.g., as a cover scent when the product is wetted would be more desirable then the smell associated with urine). By incorporating the scent into the compressed substrate it can be largely sequestered when the substrate is dry. Upon wetting, the scent becomes exposed to the environment, and therefore more perceptible as the material expands (increasing the surface area). Examples of scents that could be sequestered include floral scents (rose, lilac or lavender), clean or clean/soapy type scents associated with commonly sold household soaps, etc. These scents would preferably be in water soluble form, but could alternatively be in oil based carrier(s). Thus, these olfactory cues can be cover scents as listed above or cue to the wearer or caregiver that an incontinent event has occurred.

### IV. Absorbent Articles

The compressed substrate 10 can be included in an absorbent article as a tactile cue to indicate to the wearer that an insult has occurred. Upon wetting, the compressed substrate 10 can expand to press against the wearers skin. The term “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, etc.), and an absorbent core.

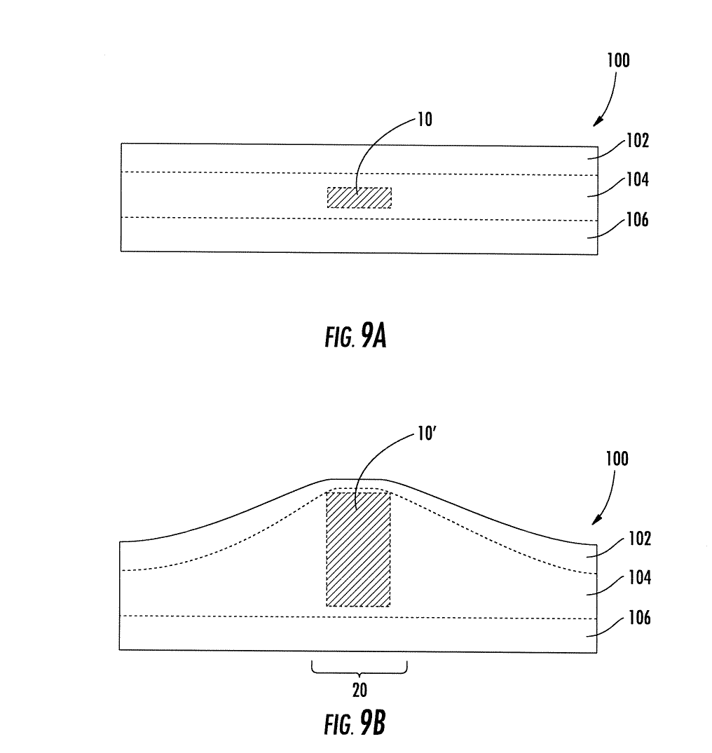

With particular reference now to FIG. 9A, a compressed substrate 10 is suitably disposed between the liquid-permeable layer 102 and the liquid-impermeable layer 106 so that the compressed substrate 10 is substantially imperceptible to the wearer prior to the first insult of the absorbent article 100 by liquid body exudates (e.g., urine, menses, feces).

The compressed substrate 10 can be positioned in the crotch region of the absorbent article (e.g., within the middle third of the absorbent article in both the longitudinal and lateral directions). However, it is contemplated that the longitudinal position of the compressed substrate 10 within the crotch region (e.g., the middle third of the length of the absorbent article) may be dependant on the type of absorbent article and/or the gender of the intended wearer.

While a single compressed substrate 10 is shown in the illustrated embodiment of FIG. 9A, it is contemplated that additional compressed substrates 10 may be used to further enhance the signal to the wearer. For example, additional compressed substrates 10 may be necessary for larger absorbent articles for whom the resistive force provided by a single compressed substrate 10 may be insufficient to alert the wearer to insult of the absorbent article 100.

The thickness, or height H, of the compressed substrate 10 when dry is suitably in the range of about 2 mm to about 20 mm, and more suitably in the range of about 5 mm to about 15 mm, such as about 10 mm. Upon absorption of a liquid, the thickness, or height H′, of the expanded compressed substrate 10′ suitably expands to at least about 2 times its original height H when dry, and more suitably it expands to at least about 3 times the height H when dry. For example, in some embodiments, the expanded compressed substrate 10′ can have a thickness or height H′ that is from about 5 times to about 10 times its original height H. This 1-dimensional expansion is generally achieved according to the expansion ratio described above, with contact of greater than 0.1 mL of a liquid.

At the relatively small initial height H, the compressed substrate 10 does not substantially interfere with the flexibility of the absorbent article, nor does the compressed substrate 10 substantially interfere with the absorbent capacity of the absorbent core 16. For example, the compressed substrate 10 can have a width of less than about 33% of the width of the absorbent core, such as less than about 25%. In most embodiments, the compressed substrate 10 has a width and length in the x- and y-directions of less than about 5 centimeters (cm), such as from about 1 cm to about 4 cm, and from about 2 cm to about 3 cm.

Various embodiments of an absorbent article that may be formed according to the present invention, such as diapers, incontinence articles, sanitary napkins, diaper pants, feminine napkins, children's training pants, and so forth. Various configurations of a diaper are described in U.S. Pat. No. 4,798,603 to Meyer et al.; U.S. Pat. No. 5,176,668 to Bemardin; U.S. Pat. No. 5,176,672 to Bruemmer et al.; U.S. Pat. No. 5,192,606 to Proxmire et al.; and U.S. Pat. No. 5,509,915 to Hanson et al., as well as U.S. Patent Application Pub. No. 2003/120253 to Wentzel, et al., all of which are incorporated herein in their entirety by reference thereto for all purposes.

In another embodiment, a training pant can be constructed with a compressed substrate 10 within the crotch region. The training pant can have a similar construction than the diaper described above. As stated, the compressed substrate 10 of the illustrated embodiment is small enough to not take up a substantial part of the crotch region. In another embodiment, the compressed substrate 10 can be included within a sanitary napkin for feminine hygiene.

### EXAMPLES

### Example 1

### Concave Upper Surface

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). This compressed substrate was cut using a sharp knife to form a hole in the top surface. The hole made was cone shaped and about 1 cm in depth. When the compressed substrate was placed on a plate and water poured into the plate the compressed substrate expanded rapidly in the z-direction. The top portion of the compressed substrate also expanded to a thimble shape with the sides rising to make the thimble. The final expanded size was 3.8 cm tall, and the hole expanded to a depth of 1.8 cm deep (thimble depth) and had a hole diameter of 1.5 cm at the upper most point.

### Example 2

### Convex Upper Surface

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). This compressed substrate was cut using a razor blade to form a cone shape (convex) on the top by cutting away material from the edges around the top surface. The cone was 0.5 cm in height with a 1 cm base. The shaped compressed substrate was then placed on a plate and water poured onto the plate. The compressed substrate rapidly expanded in the z-direction to yield an expanded substrate having a final total height of 4 cm tall, which includes a 2 cm tall cone shape.

### Example 3

### Two Linear Cuts having a Cross-Like Shape in the Upper Surface

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). This compressed substrate was cut twice using a razor blade to form 2 linear cuts in a cross-like manner. The linear cuts were substantially perpendicular to each other and crossed each other at about the middle point of the compressed substrate. Each linear cut was about 0.25 cm in depth. When the compressed substrate was placed on a plate and water poured onto the plate, the compressed substrate rapidly expanded in the z-direction manner. When the liquid reached the upper portion that encompassed the cut areas, the upper portion expanded upward and outwards generating a crown tooth shape. The final height of the expanded substrate was 4.4 cm, with a diameter of 2 cm at the base and 3 cm across at the uppermost portion of the crown section.

### Example 4

### Flowering Expansion of the Upper Surface

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). Three linear cuts were made using a razor blade. These cuts were substantially equal-distance apart from each other and through the center of the upper surface. The cuts divided the upper surface of the compressed substrate into six “pie slices” that are each about the same size. Each cut was made to a depth of 1 cm into the compressed substrate. When this cut compressed substrate was placed on a plate and water poured into the plate the compressed substrate expanded in the z-direction until the cuts were reached by the water. As the water wicked up the compressed substrate, the cut sections of the upper half bloomed open expanded outwards to form the structure of a “carnation-like” flower where each sheet of the compressed substrate opened up as a petal of the flower. While the base remained 2 cm in diameter, the opened cut section expanded in the x-y direction to about 5 cm in diameter and 2 cm in height.

### Example 5

### Linear Cuts having a V-Notch Shape Upper Section

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). This compressed substrate was cut twice using a razor blade to form 2 linear cuts oriented in a v-notch shape. The depth of each notch was 0.5 cm deep, and the cuts had a distance of 2 cm at the widest top section. The compressed substrate was then placed on a plate and water poured onto the plate. The compressed substrate rapidly expanded in the z-direction until it reached the v-notch which expnded upward and outwards. The final shape had a 2 cm diameter based with a rabbit ear-like top section measuring 3.5 cm across and 2 cm deep. The base section was 2 cm in height.

### Example 6

### Chisel-Shaped Upper Section

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). This compressed substrate was cut using a razor blade to form a chisel shape (i.e., a linear protrusion across the diameter of the upper surface) by cutting off two wedges from the center to the outer edge of the compressed substrate. The shaped section was 0.5 cm in height with 0.5 cm wide base. The compressed substrate was then placed on a plate and water poured onto the plate. The compressed substrate expanded rapidly in the z-direction. The chisel shape expanded to 2 cm in height with a 2 cm wide base.

### Example 7

### Cooling Active Agent

0.5 g of xylitol powder was sprinkled over the center section a 20 cm×20 cm sample of spunlace web (100% rayon DuPont spunlace fabric). The fabric was then folded together, and then rolled to yield a cylinder 6 cm long and 2 cm diameter. The roll was then compressed to 14,000 lb pressure using a Carver hydraulic press (Model Auto Series 3896-4D10A00, Carver Inc., Wabash Ind.) at ambient temperature. The compressed rectangle obtained was 6 cm long and 0.5 cm thick. When placed in tray and wet with water the compressed shape expanded and felt quite cold to the touch due to the cooling effect of the xylitol.

### Example 8

### Odor Adsorbing Active Agent

1 g of activated carbon powder (Nuchar WV-B1500, MeadWestvaco, Charleston, S.C.) was sprinkled onto the center section a 20 cm×20 cm sample of spunlace web (100% rayon DuPont spunlace fabric). The fabric was then folded and rolled into a cylinder shape and placed into a stainless steel mold. The mold was shaped to make a shape similar to a tongue depressor. The mold was placed into the Carver press and 14,000 lb pressure applied to the mold. The compressed fabric containing the activated carbon powder was 8 cm long×3 mm thick and 1.5 cm wide at the ends reaching 2 cm wide at the center section. When wet with water the shape expanded rapidly opening the structure to allow the activated carbon powder to be available for odor removal.

### Example 9

### Cooling Active Agent

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). A small amount of office adhesive (Elmer's Rubber cement, Elmer's Products Inc., Columbus Ohio) was placed on the upper surface of a compressed substrate using of the brush applicator. Powdered xylitol (0.4 g Aldrich Chemical Company, Milwaukee Wis.) was sprinkled over this thin adhesive coating and adhered to the adhesive. When the compressed substrate was placed on a plate and water onto the plate the pill expanded rapidly in the z-direction. When the fluid reached the top of the compressed substrate it became cold to the touch. This illustrates the potential for the compressed substrate to deliver the active (e.g. cooling, warming, etc.) to the skin or body part of the user who is wearing the article to alert them that the diaper is wet or reaching capacity or simple training purposes.

### Example 10

### Delivery of Agent through the Center of the Compressed Substate

A spunlace nonwoven web (100% rayon DuPont spunlace fabric) was compressed into a compressed substrate having dimensions of about 1 cm in height and about 2 cm in diameter (the compressed substrate was provided by COSCO International, Seoul, South Korea). Two drops (25 μL) acetone solution (10 mg/ml) of Drug & Cosmetic (D&C) Red 27 (Noveon Hilton Davis, Inc., Cincinnati Ohio) was applied onto the upper surface of the compressed substrate and allowed to dry. This dyed end was placed down on the plate and water poured onto the plate. The pill expanded in the z-direction. No dye was observed on the outside of the expanded pill, but dye was observed to appear on the top of the expanded pill opposite the upper surface where the dye was applied. Upon unrolling the expanded spunlace web, the dye could be seen in the core section of the compressed substrate as it was transported by the fluid wicking up the center of the compressed fabric in the z-direction. This example illustrates the potential for actives to be transported from one end of the compressed object to be delivered to the other end. Or in addition, transported from the center of the compressed pill to one end. Interestingly, no dye was observed on the sides of the expanding pill, showing no wicking in the x-y direction.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. An absorbent article configured to transfer an active agent to a wearer, the absorbent article comprising:
a liquid-permeable layer;
a liquid-impermeable layer;
an absorbent core positioned between the liquid-permeable layer and the liquid-impermeable layer; and
a compressed substrate positioned between the liquid-permeable layer and the liquid-impermeable layer, wherein the compressed substrate defines a x-direction, a y-direction, a z-direction, and an altered upper surface, the altered upper surface defining an initial surface area, wherein the compressed substrate comprises a compression molded web and an active agent, wherein the compressed substrate is configured to expand in the z-direction upon contact with a liquid to form an expanded substrate without substantially expanding in either the x-direction or the y-direction, and wherein the altered upper surface of the expanded substrate has an expanded surface area that is at least about 110% of the initial surface area of the compressed substrate.

2. An absorbent article as in claim 1, wherein the altered upper surface comprises a concave upper surface.

3. An absorbent article as in claim 1, wherein the altered upper surface comprises a plurality of apertures.

4. An absorbent article as in claim 1, wherein the altered upper surface comprises a convex upper surface.

5. An absorbent article as in claim 1, wherein the altered upper surface comprises a plurality of protuberances.

6. An absorbent article as in claim 1, wherein the altered upper surface comprises at least one linear cut.

7. An absorbent article as in claim 6, wherein the altered upper surface comprises three linear cuts extending across the length of the upper surface, wherein the three linear cuts divide the upper surface into six substantially equally sized regions.

8. An absorbent article as in claim 1, wherein the compressed substrate is configured to at least double in size in the z-direction upon contact with a liquid.

9. An absorbent article as in claim 1, wherein the compressed substrate is configured to at least triple in size in the z-direction upon contact with a liquid.

10. An absorbent article as in claim 1, wherein the compressed substrate is configured to expand from about 5 times to about 10 times of its size in the z-direction upon contact with a liquid.

11. An absorbent article as in claim 1, wherein the compressed substrate is configured to expand only up to about 110% of its original size in both the x-direction and the y-direction.

12. An absorbent article as in claim 1, wherein the compression molded web comprises a nonwoven web of pulp staple fibers.

13. An absorbent article as in claim 1, wherein the active agent comprises a physiological cooling agent.

14. An absorbent article as in claim 1, wherein the active agent comprises a fizzing agent.

15. An absorbent article as in claim 1, wherein the active agent comprises a beneficial agent configured to provide a benefit to the wearer.

16. An absorbent article as in claim 1, wherein the active agent comprises activated carbon.

17. An absorbent article as in claim 1, wherein the active agent comprises an olfactory agent.

18. A method of altering an upper surface of a compressed substrate, the method comprising:
providing a compressed substrate defining an upper surface having an initial surface area, wherein the compressed substrate comprises a compression molded web and an active agent, wherein the compressed substrate has an expansion ratio of greater than about 2:1.1; and
altering the upper surface of the compressed substrate such that upon contact with a liquid, the altered upper surface of the expanded substrate has an expanded surface area that is at least about 110% of the initial surface area of the compressed substrate.

19. A method of making a compressed substrate having an altered upper surface, the method comprising:
positioning a web material into an elongated barrel;
subjecting the web material to a compression force in a direction of the elongation of the barrel, wherein the compression force is provided by moving a pressing rod through the elongated barrel, wherein the pressing rod has a contact surface configured to alter the upper surface of the formed compressed substrate.

20. A compressed substrate defining a x-direction, a y-direction, a z-direction, and an altered upper surface, the altered upper surface defining an initial surface area, the compressed substrate comprising a compression molded web and an active agent, wherein the compressed substrate is configured to expand in the z-direction upon contact with a liquid to form an expanded substrate without substantially expanding in either the x-direction or the y-direction, and wherein the altered upper surface of the expanded substrate has an expanded surface area that is at least about 110% of the initial surface area of the compressed substrate.
